(19) United States
(12) Patent Application Publication
(10) Pub. No.: US 2021/0033570 A1
(43) Pub. Date: Feb. 4, 2021

(54) Method of Diagnosing Oil-Immersed Electrical Apparatus

(71) Applicant: Mitsubishi Electric Corporation, Chiyoda-ku, Tokyo (JP)

(72) Inventors: Fukutaro Kato, Chiyoda-ku, Tokyo (JP); Satoru Toyama, Chiyoda-ku, Tokyo (JP)

(73) Assignee: Mitsubishi Electric Corporation, Chiyoda-ku, Tokyo (JP)

(21) Appl. No.: 16/964,267

(22) PCT Filed: Apr. 25, 2018
(86) PCT No.: PCT/JP2018/016801

§ 371 Date: Jul. 23, 2020

(51) Int. Cl. G01N 30/06; G01N 33/03; G01N 33/34

(52) U.S. Cl. CPC G01N 30/06; G01N 33/34; G01N 33/03

(57) ABSTRACT

The present invention provides a method of diagnosing an oil-immersed electrical apparatus by an assessment of a state of deterioration of the oil-immersed electrical apparatus including an insulating oil. The insulating oil has been subjected to changing from mineral oil to vegetable oil. The method comprises: performing a first analysis before the changing, by analyzing the insulating oil; performing a second analysis after the changing, by analyzing the insulating oil; performing a first assessment by assessing the state of deterioration of the oil-immersed electrical apparatus before the changing, based on the first analysis results; performing a second assessment by assessing the state of deterioration of the oil-immersed electrical apparatus after the changing, based on the second analysis results; and performing a third assessment by assessing the state of deterioration of the oil-immersed electrical apparatus based on assessment results from the first assessment and assessment results from the second assessment.

### TECHNICAL FIELD

The present invention relates to a method of diagnosing an oil-immersed electrical apparatus.

### BACKGROUND ART

When oil-immersed electrical apparatuses such as transformers deteriorate in which an insulating paper and a copper coil, etc. are placed in electrically insulating oil (insulating oil), it is mainly due to the deterioration of the insulating paper. A diagnosing method which analyzes a deterioration marker substance produced in the insulating oil as the insulating paper deteriorates and thereby assesses the state of deterioration of the oil-immersed electrical apparatus (or the insulating paper) is known (for example, PTL 1).

As the insulating oil of oil-immersed electrical apparatuses, mineral oil has been mainly used conventionally. In recent years, however, due to the growing awareness of disaster prevention and environmental friendliness, there is an increasing trend of application of vegetable oil to oil-immersed electrical apparatuses. In addition, because vegetable oil has a higher saturated moisture content than that of mineral oil, moisture in the insulating paper moves into the insulating oil (vegetable oil) and thereby hydrolysis of the insulating paper may be reduced, which is another advantage of vegetable oil. For these reasons, changing the insulating oil from mineral oil to vegetable oil is expected to increase the life of the oil-immersed electrical apparatus compared to using mineral oil (see the upper graph in FIG. 2).

Application of vegetable oil to oil-immersed electrical apparatuses has been practiced not only in newly established oil-immersed electrical apparatuses but also in existing oil-immersed electrical apparatuses. In other words, changing conventionally-used insulating oil (mineral oil) to vegetable oil in existing oil-immersed electrical apparatuses (retrofit) is practiced (for example, NPL 1).

### CITATION LIST

### Patent Literature

PTL 1: Japanese Patent Publication No. 6-48654

### Non Patent Literature

NPL 1: K. J. Rapp, J. Luksich and A. Sbravati, Application of Natural Ester Insulating Liquids in Power Transformers, Proceedings of My Transfo, Italy, 2014.

### SUMMARY OF INVENTION

### Technical Problem

However, when the insulating oil is changed from mineral oil to vegetable oil, a part of the deterioration marker substance adhered to the insulating paper before the changing during operation with mineral oil moves into the vegetable oil, in which no deterioration marker substance is dissolved, after the changing. This deterioration marker substance that moved from the insulating paper into the vegetable oil serves as an error factor, making it difficult to accurately assess the state of deterioration of the oil-immersed electrical apparatus based on analysis results for the deterioration marker substance in the insulating oil after the changing of the insulating oil, which is a problem.

Therefore, an object of the present invention is to provide a method of diagnosing an oil-immersed electrical apparatus that makes it possible to accurately assess the state of deterioration of an oil-immersed electrical apparatus in which insulating oil has been subjected to changing from mineral oil to vegetable oil.

### Solution to Problem

The present invention provides a method of diagnosing an oil-immersed electrical apparatus by an assessment of the state of deterioration of the oil-immersed electrical apparatus.

The oil-immersed electrical apparatus includes an insulating oil and an insulating paper. The insulating oil has been subjected to changing from mineral oil to vegetable oil.

The method of diagnosing an oil-immersed electrical apparatus according to the present invention comprises:

performing a first analysis before the changing, by analyzing a deterioration marker substance in the mineral oil in the oil-immersed electrical apparatus;

performing a second analysis after the changing, by analyzing a deterioration marker substance in the vegetable oil in the oil-immersed electrical apparatus;

performing a first assessment by assessing the state of deterioration of the oil-immersed electrical apparatus as of a point in time before the changing, based on analysis results from the first analysis;

performing a second assessment by assessing the state of deterioration of the oil-immersed electrical apparatus as of a point in time after the changing, based on analysis results from the second analysis, and also based on an estimated amount of increment of the deterioration marker substance caused by a movement of a part of the deterioration marker substance once adhered to the insulating paper before the changing and then moved into the vegetable oil after the changing; and

performing a third assessment by assessing the state of deterioration of the oil-immersed electrical apparatus based on assessment results from the first assessment and assessment results from the second assessment.

### Advantageous Effects of Invention

The present invention provides a method of diagnosing an oil-immersed electrical apparatus capable of accurately assessing the state of deterioration of the oil-immersed electrical apparatus in which insulating oil has been subjected to changing from mineral oil to vegetable oil.

### BRIEF DESCRIPTION OF DRAWINGS

FIG. 1 is a flowchart illustrating an example method of diagnosing an oil-immersed electrical apparatus according to an embodiment 1.

FIG. 2 is a conceptual view illustrating an example method of diagnosing an oil-immersed electrical apparatus according to embodiments 1 to 3.

FIG. 3 is a graph showing the correlation between the furfural concentration of an insulating oil used in a method of diagnosing an oil-immersed electrical apparatus according to an embodiment 4 and the average degree of polymerization of an insulating paper.

FIG. 4 is a conceptual view illustrating a method of extracting a deterioration marker substance in an insulating oil by solid-phase micro-extraction and analyzing the same, for use in a method of diagnosing an oil-immersed electrical apparatus according to an embodiment 6.

FIG. 5 is a diagram illustrating the configuration of an oil-immersed electrical apparatus diagnosis system according to an embodiment 8.

### DESCRIPTION OF EMBODIMENTS

In the following, a description will be given of embodiments of the present invention referring to drawings. In the drawings of the present invention, the same or equivalent members are denoted by the same reference numerals. Dimensions in the drawings, including length, width, thickness, and depth, may have been changed from the actual dimensions as appropriate for the clarification and simplification of the drawings, and therefore, dimensions may not agree with the actual dimensions.

### Embodiment 1

A method of diagnosing an oil-immersed electrical apparatus according to the present embodiment assesses the state of deterioration of an oil-immersed electrical apparatus.

The oil-immersed electrical apparatus is not particularly limited provided that it is an electrical apparatus including an insulating oil. In the oil-immersed electrical apparatus, an electric insulator and a conductor are preferably immersed in the insulating oil. The oil-immersed electrical apparatus is preferably a transformer.

In the oil-immersed electrical apparatus that is a subject of the diagnosis according to the present embodiment, the insulating oil has been subjected to changing from mineral oil to vegetable oil. In other words, the diagnosis method according to the present embodiment is for an oil-immersed electrical apparatus including vegetable oil as insulating oil which once included mineral oil as insulating oil before the changing.

The “state of deterioration of an(the) oil-immersed electrical apparatus” refers to, for example, the degree of deterioration of the oil-immersed electrical apparatus. By assessing the state of deterioration of the oil-immersed electrical apparatus and, then, using the correlation between the state of deterioration of the oil-immersed electrical apparatus and the remaining life of the oil-immersed electrical apparatus, estimation of the remaining life of the oil-immersed electrical apparatus may be carried out. The estimation of the remaining life makes it possible to propose a timely update of the oil-immersed electrical apparatus.

In the diagnosis method according to the present embodiment, from assessment results of the state of deterioration of the oil-immersed electrical apparatus (or the insulating paper) as of each of the time period during which the electrical apparatus including mineral oil is operated before changing the insulating oil from mineral oil to vegetable oil (hereinafter, sometimes simply called “changing”) and the time period during which the electrical apparatus including vegetable oil is operated after the changing, an overall assessment is made.

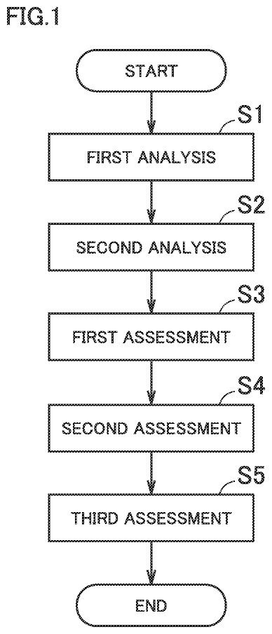

More specifically, referring to FIG. 1, the method of diagnosing an oil-immersed electrical apparatus according to the present embodiment comprises a first analysis (S1), a second analysis (S2), a first assessment (S3), a second assessment (S4), and a third assessment (S5), as described below. It is not necessary to perform S1 to S5 in this order provided that S1 is performed before S3, S2 is performed before S4, and both S3 and S4 are performed before S5.

(First Analysis)

In this step, a deterioration marker substance in the mineral oil (insulating oil) in the oil-immersed electrical apparatus is analyzed before the changing.

The deterioration marker substance is not particularly limited, and it is, for example, a substance produced as the insulating paper(such as cellulose-containing insulating paper) deteriorates. Specific examples of the deterioration marker substance include furfural, CO+CO₂, acetone, methanol, and ethanol. One type of the deterioration marker substance may be analyzed, or multiple types of the substance may be analyzed either simultaneously or separately.

(Second Analysis)

In this step, the deterioration marker substance in the vegetable oil (insulating oil) in the oil-immersed electrical apparatus is analyzed after the changing. Examples of the vegetable oil include oils containing an ester bond (ester oil).

(First Assessment)

In this step, based on analysis results from the first analysis, the state of deterioration of the oil-immersed electrical apparatus as of a point in time before the changing is assessed.

(Second Assessment)

In this step, the state of deterioration of the oil-immersed electrical apparatus as of a point in time after the changing is assessed based on analysis results from the second analysis, and also based on the estimated amount of increment of the deterioration marker substance caused by the movement of a part of the deterioration marker substance once adhered to the insulating paper before the changing and then moved into the vegetable oil after the changing.

(Third Assessment)

In this step, the state of deterioration of the oil-immersed electrical apparatus is assessed based on assessment results from the first assessment and assessment results from the second assessment.

In the diagnosis method according to the present embodiment as described above, an overall assessment of the oil-immersed electrical apparatus in which insulating oil has been subjected to changing from mineral oil to vegetable oil is made from assessment results for each of the time period during which mineral oil is used before the changing and the time period during which vegetable oil is used after the changing. For instance, the degree of deterioration from the first assessment and the degree of deterioration from the second assessment are combined, and the resulting combined degree of deterioration is regarded as the degree of deterioration of the subject oil-immersed electrical apparatus.

Further, in the diagnosis method according to the present embodiment, in the assessment for the time period during which vegetable oil is used after the changing, an increment of the deterioration marker substance caused by the movement of a part of the deterioration marker substance once adhered to the insulating paper before the changing and then moved into the vegetable oil after the changing is taken into consideration. This makes it possible to accurately assess the state of deterioration of the oil-immersed electrical apparatus (or the insulating paper).

### Embodiment 2

In this embodiment, the first analysis (S1) and the first assessment (S3), and the second analysis (S2) and the second assessment (S4), all according to embodiment 1, are carried out as follows.

(First Analysis)

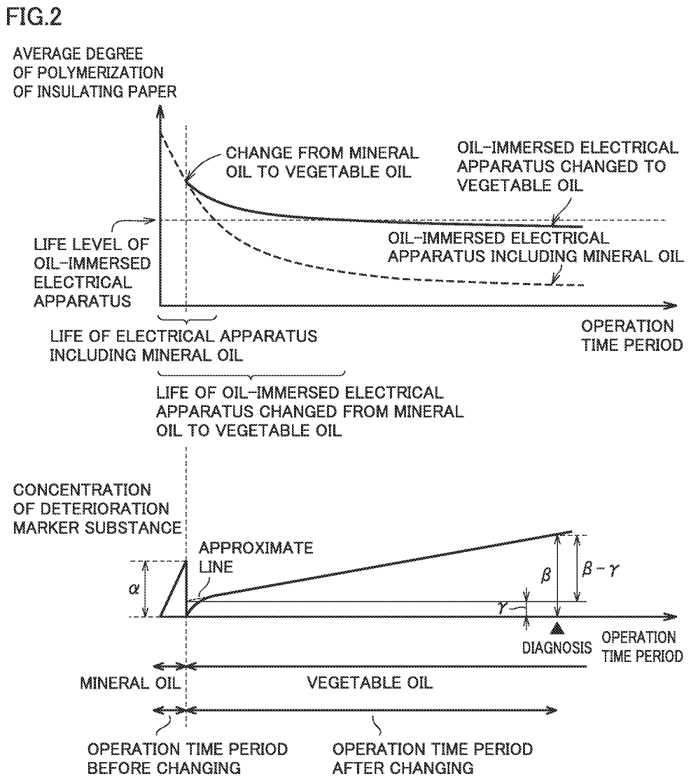

The concentration of the deterioration marker substance in the insulating oil (mineral oil) immediately before the changing to vegetable oil (a) is measured (see the lower graph in FIG. 2). In the lower graph in FIG. 2, the slope of the plot of the concentration of the deterioration marker substance for the time period of operation before the changing is very steep, and it is because the time period of operation before the changing is shown shorter in the graph than its actual length.

(First Assessment)

From the concentration of the deterioration marker substance measured in the first analysis (α), the average degree of polymerization of the insulating paper is estimated, and thereby the state of deterioration of the insulating paper is assessed. The average degree of polymerization of the insulating paper means, for example, the number of glucose groups constituting the insulating paper.

(Second Analysis)

The concentration of the deterioration marker substance in the insulating oil (vegetable oil) at the time of diagnosis after the changing (β) is measured (see the lower graph in FIG. 2).

From this concentration of the deterioration marker substance (β) thus measured, an increment of the concentration of the deterioration marker substance caused by the movement of a part of the deterioration marker substance once adhered to the insulating paper and then moved into the vegetable oil after the changing (γ) is subtracted to determine the concentration of the deterioration marker substance produced as the insulating paper deteriorated during operation of the electrical apparatus including vegetable oil after the changing (β-γ) (see the lower graph in FIG. 2).

(Second Assessment) From the concentration (β-γ) thus determined in the second analysis, the average degree of polymerization of the insulating paper is estimated, and thereby the state of deterioration of the insulating paper is assessed.

In this embodiment, in the oil-immersed electrical apparatus in which insulating oil has been subjected to changing from mineral oil to vegetable oil, the concentration of the deterioration marker substance produced in the insulating oil as the insulating paper deteriorates may be accurately estimated, and thereby the state of deterioration of the oil-immersed electrical apparatus (or the insulating paper may be accurately assessed.

### Embodiment 3

In this embodiment, the increment of the concentration of the deterioration marker substance caused by the movement of a part of the deterioration marker substance once adhered to the insulating paper and then moved into the vegetable oil after the changing, as described in embodiment 2, is obtained as follows. After the changing to vegetable oil is performed, the concentration of the deterioration marker substance is measured at least twice (preferably at least three times) with oil sampling performed at any intervals (for example, every 3 to 4 years), and the measurement results are used to obtain an approximate line (approximation expression) that approximates the trend of the concentration of the deterioration marker substance. The value on the approximate line for a point in time of the changing is regarded as the increment of the concentration of the deterioration marker substance caused by the movement of a part of the deterioration marker substance that moved from the insulating paper into the vegetable oil after the changing (γ) (see the lower graph in FIG. 2).

In this embodiment, the concentration of the deterioration marker substance that moved from the insulating paper into the vegetable oil after the changing may be estimated. By this, the concentration of the deterioration marker substance produced in the insulating oil as the insulating paper deteriorates may be accurately estimated, and thereby the state of deterioration of the oil-immersed electrical apparatus (or the insulating paper) may be accurately assessed.

### Embodiment 4

In this embodiment, as the deterioration marker substance according to embodiments 1 to 3, the furfural concentration in the insulating oil is measured. CO, CO₂, acetone, and the like can be produced upon an abnormality other than deterioration of the insulating paper and therefore the cause of its production may not always be identifiable; furfural is known to be produced directly due to the deterioration of the insulating paper and is therefore suitable for use as an accurate indicator of the state of deterioration of the insulating paper.

Sealed-type oil-immersed electrical apparatuses commonly distributed in Japan have a track record in diagnosis by furfural measurement in insulating oil. In contrast, for open-type oil-immersed transformers for distribution overseas, no deterioration marker substance has been selected.

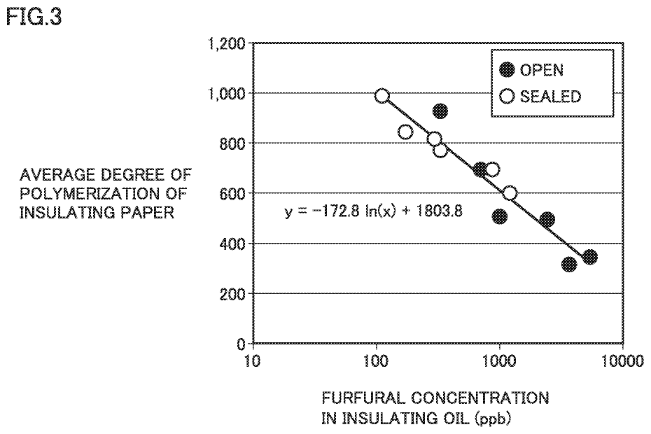

In this regard, as shown in FIG. 3, experiments conducted by the inventors of the present invention have made it clear that the correlation between the furfural concentration in insulating oil and the average degree of polymerization of the insulating paper in an oil-immersed electrical apparatus (open-type oil-immersed electrical apparatus), in which the chamber containing insulating oil and the like has space above the oil surface, agrees with the corresponding correlation in an oil-immersed electrical apparatus (sealed-type oil-immersed electrical apparatus) with no such space.

This suggests that furfural tends not to elute into the gas in the space that is in contact with insulating oil or elsewhere, and therefore, the diagnosis method according to the present embodiment may also be implemented for diagnosing an open-type oil-immersed electrical apparatus without requiring correction of the furfural concentration in insulating oil. Thus, the diagnosis method according to the present embodiment may be applicable to both an sealed-type oil-immersed electrical apparatus and an open-type oil-immersed electrical apparatus.

### Embodiment 5

In this embodiment, in the first assessment and the second assessment according to embodiment 2, in estimating the average degree of polymerization of the insulating paper from the concentration of the deterioration marker substance (or concentration difference), a mathematical relation between the concentration of the deterioration marker substance and the average degree of polymerization of the insulating paper is used. Different mathematical relations are used for mineral oil and for vegetable oil. More specifically, the mathematical relation used in estimating the average degree of polymerization of the insulating paper in the first analysis is different from the mathematical relation used in the second analysis.

Mineral oil and vegetable oil are different types of oil, and therefore they have different solubilities for the deterioration marker substance. Thus, by using different mathematical relations between the concentration of the deterioration marker substance and the average degree of polymerization of the insulating paper, the average degree of polymerization of the insulating paper may be estimated in more accuracy.

### Embodiment 6

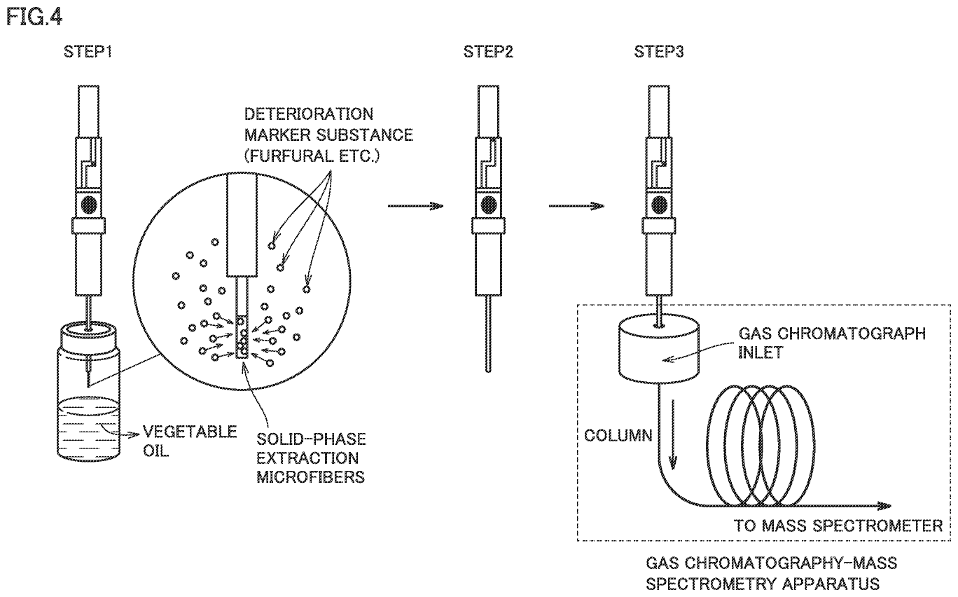

In this embodiment, in the second analysis according to embodiments 1 to 5, the deterioration marker substance (furfural, etc.) in the vegetable oil is, as shown in FIG. 4, volatilized from the vegetable oil when the vegetable oil is heated (STEP 1), adsorbed on solid-phase micro-extraction fibers (STEP 2), and then analyzed (measured) with a gas chromatography-mass spectrometry apparatus (STEP 3).

By this analysis using solid-phase micro-extraction fibers, the deterioration marker substance in the vegetable oil may be analyzed with high sensitivity without any interference from other components in the vegetable oil. This makes it possible to perform quantitative assessment up to 0.05 mg/kg required by, for example, the international standards (IEC 61198) established by the International Electrotechnical Commission (IEC).

### Embodiment 7

In this embodiment, in the first analysis according to embodiments 1 to 6, the deterioration marker substance (furfural, etc.) in the mineral oil is extracted from the mineral oil with methanol according to the Japanese standards (JPI-5S-58-99), and then analyzed in the form of methanol solution by high-performance liquid chromatography.

By this analysis with methanol extraction, the deterioration marker substance in the mineral oil may be analyzed with high sensitivity. This makes it possible to perform quantitative assessment up to 0.05 mg/kg required by, for example, the international standards (IEC 61198) established by the International Electrotechnical Commission (IEC).

### Embodiment 8

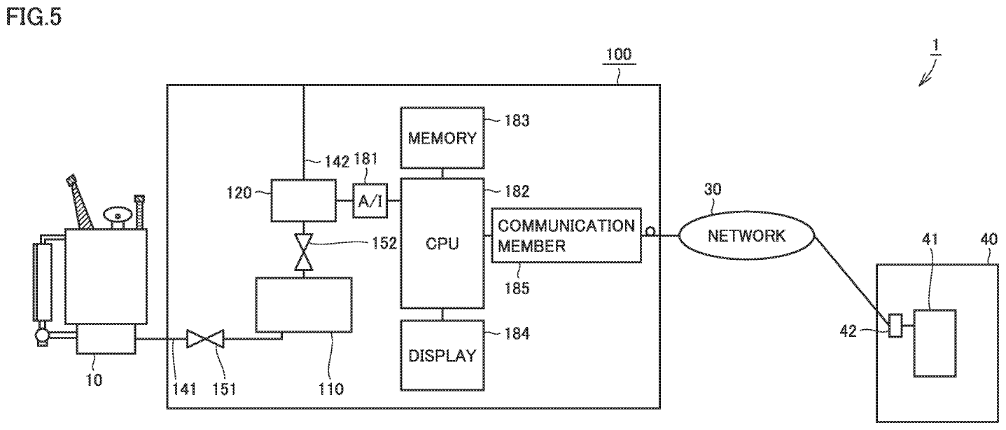

This embodiment relates to an oil-immersed electrical apparatus diagnosis system used in the method of diagnosing an oil-immersed electrical apparatus. FIG. 5 is a diagram illustrating the configuration of the oil-immersed electrical apparatus diagnosis system according to the present embodiment. The following description is for the case in which the deterioration marker substance in the insulating oil is in gas form, but the deterioration marker substance is not limited to gas.

As shown in FIG. 5, in this embodiment, a gas-in-oil analysis apparatus 100 is an apparatus for analyzing gas in insulating oil that fills inside an oil-immersed electrical apparatus 10. A first piping 141 is connected to oil-immersed electrical apparatus 10, and serves as a channel through which the insulating oil flows. First piping 141 is equipped with a first valve 151, which opens and closes first piping 141.

A gas extraction chamber 110 is connected to first piping 141. First piping 141 allows communication between oil-immersed electrical apparatus 10 and gas extraction chamber 110. Inside gas extraction chamber 110, gas is extracted from the insulating oil. For enhancing the gas extraction efficiency, gas extraction chamber 110 may be equipped with a heater capable of raising the temperature of the insulating oil to a certain temperature.

A second piping 142 is connected to gas extraction chamber 110. Second piping 142 serves as a channel through which the gas extracted from the insulating oil in gas extraction chamber 110 flows. Second piping 142 is equipped with a second valve 152, which opens and closes second piping 142. Second piping 142 is equipped with a gas detector 120. Second piping 142 allows communication between gas extraction chamber 110 and gas detector 120.

Gas detector 120 measures the concentration of each component of the gas flowing through second piping 142. Gas detector 120 is equipped with, for example, a gas separation column that separates gas into components, and a gas sensor that measures the concentration of each gas component thus separated. For example, the gas sensor may have an infrared-light-detection-type configuration that includes an infrared light source and an infrared light detector, provided that it is capable of measuring the concentration of the gas being analyzed.

Gas-in-oil analysis apparatus 100 comprises an information processor connected to gas detector 120. The information processor includes an analog signal input member 181, a CPU (Central Processing Unit) 182, a memory 183, a display 184, and a communication member 185.

Analog signal input member 181 is electrically connected to the gas sensor of gas detector 120. An analog signal output by the gas sensor of gas detector 120 is input in CPU 182 via analog signal input member 181 and converted into a gas concentration. The resulting gas concentration is stored in memory 183 and displayed on display 184.

Memory 183 also has a function of storing a program that is to be executed by CPU 182, and is composed of ROM (Read Only Memory) and RAM (Random Access Memory), for example.

In this embodiment, CPU 182 controls the combination of open/close states of first valve 151, second valve 152, and the like at the command output by the program stored in memory 183.

An oil-immersed electrical apparatus diagnosis system 1 comprises an administrative server 40 that is connected to gas-in-oil analysis apparatus 100 via a communication network 30. Administrative server 40 includes a storage 41 and a communication member 42.

The means that connects each of communication member 185 and communication member 42 to communication network 30 may be wired communication such as optical fibers or LAN (local area network) cables, or may be wireless communication on radio waves such as wireless LAN via an antenna. The means may have any configuration provided that it is an apparatus with communication function.

Communication member 42 of administrative server 40 is provided so that it may be connected to communication member 185 of gas-in-oil analysis apparatus 100 via communication network 30. The measurement results from gas detector 120, which are the gas concentration, are transmitted through communication member 185 of gas-in-oil analysis apparatus 100 and then through communication network 30 to communication member 42 of administrative server 40.

The measurement results from gas detector 120 thus transmitted to communication member 42 are stored in storage 41 of administrative server 40. The storage 41 of administrative server 40 accumulates the time course of the measurement results from gas detector 120.

In the method of diagnosing an oil-immersed electrical apparatus and the oil-immersed electrical apparatus diagnosis system according to each of the above-described embodiments, configurations that may be combined with each other may be combined with each other.

It should be construed that embodiments disclosed herein are given by way of illustration in all respects, not by way of limitation. It is intended that the scope of the present invention is defined by claims, not by the above description, and encompasses all modifications and variations equivalent in meaning and scope to the claims.

### REFERENCE SIGNS LIST

1 gas-in-oil analysis system, 10 oil-immersed electrical apparatus, 30 communication network, 40 administrative server, 41 storage, 42 communication member, 100 gas-in-oil analysis apparatus, 110 gas extraction chamber, 120 gas detector, 141 first piping, 142 second piping, 151 first valve, 152 second valve, 181 analog signal input member, 182 CPU, 183 memory, 184 display, 185 communication member.

1. A method of diagnosing an oil-immersed electrical apparatus by an assessment of a state of deterioration of the oil-immersed electrical apparatus,
the oil-immersed electrical apparatus including an insulating oil and an insulating paper,
the insulating oil having been subjected to changing from mineral oil to vegetable oil,
the method comprising:
performing a first analysis before the changing, by analyzing a deterioration marker substance in the mineral oil in the oil-immersed electrical apparatus;
performing a second analysis after the changing, by analyzing the deterioration marker substance in the vegetable oil in the oil-immersed electrical apparatus;
performing a first assessment by assessing the state of deterioration of the oil-immersed electrical apparatus as of a point in time before the changing, based on analysis results from the first analysis;
performing a second assessment by assessing the state of deterioration of the oil-immersed electrical apparatus as of a point in time after the changing, based on analysis results from the second analysis, and also based on an estimated amount of increment of the deterioration marker substance caused by a movement of a part of the deterioration marker substance once adhered to the insulating paper before the changing and then moved into the vegetable oil after the changing; and
performing a third assessment by assessing the state of deterioration of the oil-immersed electrical apparatus based on assessment results from the first assessment and assessment results from the second assessment.

2. The method according to claim 1, wherein
in the first analysis, a concentration of the deterioration marker substance in the mineral oil is measured,
in the second analysis, a concentration of the deterioration marker substance in the vegetable oil is measured, and a concentration difference is obtained by subtracting an increment of a concentration of the deterioration marker substance caused by the movement of a part of the deterioration marker substance once adhered to the insulating paper and then moved into the vegetable oil after the changing, from the concentration of the deterioration marker substance thus measured,
in the first assessment, from the concentration of the deterioration marker substance measured in the first analysis, an average degree of polymerization of the insulating paper is estimated, and thereby a state of deterioration of the insulating paper is assessed, and
in the second assessment, from the concentration difference obtained in the second analysis, an average degree of polymerization of the insulating paper is estimated, and thereby a state of deterioration of the insulating paper is assessed.

3. The method according to claim 2, wherein, in the second assessment, from measurement results of at least two measurements of the concentration of the deterioration marker substance in the vegetable oil after the changing, an approximate line that approximates a trend of the concentration of the deterioration marker substance is obtained, and a value on the approximate line for a point in time of the changing is regarded as the increment.

4. The method according to claim 1, wherein the deterioration marker substance is furfural.

5. The method according to claim 2, wherein, in the first assessment and the second assessment, in estimating the average degree of polymerization of the insulating paper, a mathematical relation between the concentration of the deterioration marker substance and the average degree of polymerization of the insulating paper is used, and the mathematical relation for the first assessment is different from the mathematical relation for the second assessment.

6. The method according to claim 1, wherein, in the second analysis, the deterioration marker substance in the vegetable oil is volatilized from the vegetable oil when the vegetable oil is heated, adsorbed on solid-phase micro-extraction fibers, and then analyzed with a gas chromatography-mass spectrometry apparatus.

7. The method according to claim 1, wherein, in the first analysis, the deterioration marker substance in the mineral oil is extracted from the mineral oil with methanol and then analyzed by high-performance liquid chromatography.
